(19) United States
(12) Patent Application Publication
(10) Pub. No.: US 2024/0139115 A1
(43) Pub. Date: May 2, 2024

(54) Novel Method for Preparation of Nanoparticles Comprising Cell Penetrating Peptides

(71) Applicant: CELLPEPT SWEDEN AB, Stockholm (SE)

(72) Inventors: Margus Pooga, Tartu (EE); Maria Maloverjan, Tartu (EE); Ana Rebane, Tartu Country (EE); Sebastian Warmlander, Stockholm (SE); Astrid Graslund, Solleftea (SE); Juri Jarvet, Sundbyberg (SE)

(21) Appl. No.: 18/574,240

(22) PCT Filed: Jun. 30, 2022
(86) PCT No.: PCT/SE2022/050657

§ 371 Date: Dec. 26, 2023

(30) Foreign Application Priority Data
Jun. 30, 2021 (SE) 2150838-7

(51) Int. Cl. A61K 9/51; A61K 45/06; A61K 47/10

(52) U.S. Cl. CPC A61K 9/5169; A61K 9/5192; A61K 45/06; A61K 47/10

(57) ABSTRACT

The present invention relates to a method of preparing a nanoparticle comprising at least one cell-penetrating peptide (CPP) and a cargo molecule, said method comprising the steps: preparing a CPP solution comprising a CPP, a dry alcohol; and 1-50% (v/v) of an aprotic solvent; diluting the CPP solution in water to a concentration of 2 to 500 μM, and adding the cargo molecule to the CPP solution to obtain a CPP:CM mixture; whereby nanoparticles comprising CPP and cargo molecule are formed. The invention further relates to a nanoparticle obtained or obtainable by the method, as well as pharmaceutical compositions comprising such nanoparticles.

### TECHNICAL FIELD

The present invention relates to the field of biotechnological tools for delivering substances of interest to biological cells. In particular, the present invention relates to new methods for preparing nanoparticles comprising cell-penetrating peptides and cargo molecules such as proteins and nucleic acids.

### BACKGROUND

Delivering medical drugs to the right location in the body has been a long-standing problem in medical practice. Different techniques for drug delivery have been developed, including the use of cell-penetrating peptides. Such peptides have the capacity to cross biological membranes, such as cell walls and the blood-brain-barrier, and may also bring cargo molecules (i.e., drugs) with them when they cross these membranes. In some situations, using such delivery peptides can be a useful technique for drug delivery.

Cell-penetrating peptides (CPP) are capable of delivering into cells different types of macromolecules, including proteins, peptides, oligonucleotides with various activity (mRNA, siRNA, miRNA, antisense etc.), plasmid DNA and many others. Therefore, CPPs enable regulation of processes inside cells, e.g. by directly modulating expression of target genes by switching these on, knocking down, modulating activity etc.

The most efficient cell-penetrating peptides (CPPs) used for delivery of nucleic acids into cells in vitro and in vivo are PepFects (PFs) and NickFects (NFs) (1) (2). Both types contain a hydrophobic modification, as a fatty acid residue (C16-C22) has been attached to their N-terminus³. Dissolving PFs or NFs in water induces formation of large elongated micelles and other peptide aggregates. Mixing such solutions with nucleic acids or other cargo molecules produces cell-penetrating nanoparticles.

### SUMMARY OF THE INVENTION

In a first aspect, the present invention relates to a method of preparing a nanoparticle comprising at least one cell-penetrating peptide (CPP) and a cargo molecule (CM), said method comprising the steps:
Preparing a CPP solution comprising a CPP, a dry alcohol; and 1-50% (v/v) of an aprotic solvent; and
Optionally diluting the CPP solution in water to a concentration of CPP of 2 to 500 μM,
Adding the cargo molecule to the CPP solution to obtain a CPP:CM mixture;
   whereby nanoparticles comprising CPP and cargo molecule are formed.

Having prepared the CPP solution comprising a CPP, a dry alcohol; and 1-50% (v/v) of an aprotic solvent, there is an optional step of diluting the CPP solution in water to a concentration of CPP of 2 to 500 μM, preferable 5-200 μM. If the concentration of the CPP solution already is in this range, dilution is obviously not needed.

At lower concentrations, the nanoparticles may not assemble properly. At higher concentrations, the concentration of organic solvent remains higher than optimal and the solution that contains nanoparticles may harm living cells in culture or experimental animal.

The water with which the CPP solution may be diluted may be pure water, or deionised water such as Milli-Q quality water.

CPPs have the capacity to cross biological membranes, such as cell walls and the blood-brain-barrier, and may also bring cargo molecules into the cells. Such cargo molecules may be different types of macromolecules, including proteins, peptides, oligonucleotides with various activity (mRNA, siRNA, miRNA, antisense, splicing, switching, etc.), plasmid DNA and many others. When the cargo consists of nucleic acids, nucleic acids of very different sizes can be used. The nucleic acid can be a small oligonucleotide, a very large plasmid, or any size in between.

Therefore, CPPs enable regulation of processes inside cells, e.g. by directly modulating expression of target genes by switching these on, knocking down, modulating activity etc.

In one embodiment, the aprotic solvent is selected from dimethyl sulfoxide (DMSO), dimethyl formamide (DMF), and tetrahydrofuran (THF).

The CPP may be a membranophilic CPP selected from a CPP having 70-100% hydrophobic amino acids, an amphipathic CPP having 45-75% hydrophobic amino acids, a CPP with an added C10-C24 fatty acid, a CPP with an added fluorinated C10-C24 fatty acid, and/or a CPP with an added cholesterol.

The C10-C24 fatty acid, the fluorinated C10-C24 fatty acid, or the cholesterol may for example be added at the N-terminus of the CPP.

Cell-penetrating peptides are divided into three classes based on their physical-chemical properties: (i) polycationic, (ii) amphipathic and (iii) hydrophobic CPPs (17, 18).

The hydrophobic CPP class contains peptides with 70-100% hydrophobic amino acids (19), i.e. non-polar amino acids with a hydropathy index >−1, such as aliphatic (valine, isoleucine, leucine, alanine) and aromatic (phenylalanine, tyrosine, tryptophan) amino acids—The amphipathic class contains peptides with both hydrophobic and cationic amino acids that organise into the respective domains/faces. Amphipathic CPPs that efficiently deliver cargos into cells contain in their sequence 45-75% hydrophobic amino acids. In the following, we consider such CPPs from the amphipathic and hydrophobic classes to be membranophilic CPPs. We also consider CPPs with hydrophobic modifications, such as added fatty acids (C10-C24) added at the N-terminus, to be membranophilic CPPs. For example, the CPPs PepFects (PFs) and NickFects (NFs) (1) (2) both contain a hydrophobic modification, as a fatty acid residue (C16-C22) has been attached to their N-terminus (3). Further, a CPP with a fluorinated C10-C24 fatty acid added at the N-terminus, a CPP with an aromatic residue added at the N terminus, and a CPP with a cholesterol added at the N-terminus are also membranophilic CPPs.

In one embodiment, the CPP solution further comprises 0.1-5% (v/v) of an organic cyclic carbonate.

Alternatively, further comprising 1-5% (v/v) of an organic cyclic carbonate.

In one embodiment, the organic cyclic carbonate is selected from the group consisting of ethylene carbonate; propylene carbonate; ethyl methyl carbonate (EMC), glycerol carbonate (4-(Hydroxymethyl)-1,3-dioxolan-2-one), and trimethylene carbonate (1,3-dioxan-2-one).

In one embodiment, the cargo molecule is a negatively charged molecule. Such a negatively charged molecule may be a nucleic acid molecule that has intrinsic biological activity in cells.

In one embodiment, the method further comprises adding a divalent metal ion to the CPP solution or the CPP:CM mixture, or to the nanoparticle.

In one embodiment, the divalent metal ion is Ca²⁺ or Mg²⁺.

Divalent metal cations may boost cellular delivery of antisense oligonucleotides by cell penetrating peptides. (20)

In one embodiment, the dry alcohol is selected from the group consisting of methanol, ethanol, 1-propanol, 1-butanol, 1-pentanol, 1-hexanol, 2-propanol, 2-butanol, 2-pentanol, 2-hexanol, tert-butanol, and 2-ethyl 1-hexanol.

In one embodiment, the dry alcohol is a branched chain alcohol.

In one embodiment, the ratio CPP:CM in the CPP:CM mixture is in the range 10:1 to 1:1, preferably 5:1.

Alternatively, the N/P (charge) ratio of CPP:CM in the CPP:CM mixture is in the range 0.3:1 to 5:1, preferably 2:1.

N means the number of amino groups in the side-chains of the peptide (i.e positively charged groups). P is the number of phosphates in nucleic acid that give the negative charge. N/P has analogous meaning with charge (peptide to nucleic acid) ratio, reflecting the charge of nanoparticle.

CPP:CM nanoparticles that are assembled using the same N/P ratio have different charge at different pH (due to protonation/deprotonation of cationic amino acids and phosphate groups).

In a further aspect, the invention relates to a nanoparticle comprising a cell-penetrating peptide and a cargo molecule, said nanoparticle being obtainable by a method according to the invention.

The cell-penetrating peptide may be a CPP that translocates into mammalian cells and delivers there cargo molecule in an active state,

In a further aspect, the invention relates to a pharmaceutical composition comprising a nanoparticle obtained with the method according to the invention, or the nanoparticle being obtainable by a method according to the invention.

### BRIEF DESCRIPTION OF THE FIGURES

FIG. 1. Comparison of the SCO cellular delivery efficiency by PF14 CPP dissolved in different solvents. Luminescence signal of reporter HeLa pLuc705 cells treated with splicing correcting 100 nM oligonucleotide (SCO-705) or its complexes with 500 nM PF14 for 24 h. PF14 was dissolved at 1 mM concentration in Milli-Q quality water or a mixture of organic solvents (DMSO/methanol 1/1, v/v). The correction of aberrant splicing by SCO increased luciferase activity (Kole's assay) that was quantified by luminescence intensity (relative luminescence units). As comparison, the effect of 200 nM SCO-705 in complexes with 1000 nM PF14 was used.

FIG. 2. Comparison of the SCO cellular delivery efficiency by PF14 CPP dissolved in different solvents. Fluorescence signal of reporter HeLa EGFP-654 cells treated with splicing correcting 100 nM oligonucleotide (SCO-654) or its complexes with 500 nM PF14 for 24 h. PF14 was dissolved at 1 mM concentration in Milli-Q quality water or a mixture of organic solvents (DMSO/methanol 1/1, v/v). The correction of aberrant splicing by SCO increased EGFP expression (Kole's assay) that was quantified as percentage of fluorescent cells in population (%). Inclusion of calcium ions into SCO-PF14 nanoparticles increases the efficiency of splicing correction.

FIG. 3. Transfection of human primary keratinocytes with EGFP-coding mRNA cargo using NF71 and PF14 CPPs.

The complexes of 200 ng of mRNA with 520 nM CPP were added into culture medium above cells per well of 24-well plate for 20 h. For the delivery and expression of EGFP, mRNA complexes were assembled with NF71 (dissolved at 1 mM concentration in ethanol/DMSO/trimethylene carbonate 78/20/2) or PF14 (PF14o—dissolved at 1 mM concentration in ethanol/DMSO, or in water—PF14w).

FIG. 4. Effect on splicing correction by nanoparticles involving cargo nucleotide SCO-705 and differently dissolved PepFect14 (PF14) CPP.

1 mM stock solutions of PF14 were prepared by dissolving the peptide in different solvents and their mixtures (proportions are given as v:v). Nanoparticles of 100 nM SCO-705, 500 nM PF14 and 3 mM MgCl2 or CaCl2 were prepared by mixing the components in MQ water. After 30 min incubation, solutions were diluted 10-fold with cell culture medium and added to HeLa pLuc 705 cells. Luciferase activity was measured after 24 h of incubation. As a negative control, cells were incubated with a medium containing 10% (v:v) of MQ water (untreated cells). In the graph the numbers on the X-axis represent: 1: Untreated cells; 2: SCO; 3a: SCO+PF14 (PF14 dissolved in water); 3b: SCO+PF14+3 mM Mg (PF14 dissolved in water); 3c: SCO+PF14+3 mM Ca (PF14 dissolved in water); 4a: SCO+PF14 (PF14 dissolved in DMF); 4b: SCO+PF14+3 mM Mg (PF14 dissolved in DMF); 4c: SCO+PF14+3 mM Ca (PF14 dissolved in DMF); 5a: SCO+PF14 (PF14 dissolved in DMF10/EtOH90), 5b: SCO+PF14+3 mM Mg (PF14 dissolved in DMF10/EtOH90); 5c: SCO+PF14+3 mM Ca (PF14 dissolved in DMF10/EtOH90); 6a: SCO+PF14 (PF14 dissolved in DMF10/iPrOH90); 6b: SCO+PF14+3 mM Mg (PF14 dissolved in DMF10/iPrOH90); 6c: SCO+PF14+3 mM Ca (PF14 dissolved in DMF10/iPrOH90); 7a: SCO+PF14 (PF14 dissolved in THF); 7b: SCO+PF14+3 mM Mg (PF14 dissolved in THF); 7c: SCO+PF14+3 mM Ca (PF14 dissolved in THF); 8a: SCO+PF14 (PF14 dissolved in THF10/EtOH90); 8b: SCO+PF14+3 mM Mg (PF14 dissolved in THF10/EtOH90); 8c: SCO+PF14+3 mM Ca (PF14 dissolved in THF10/EtOH90); 9a: SCO+PF14 (PF14 dissolved in THF10/iPrOH90); 9b: SCO+PF14+3 mM Mg (PF14 dissolved in THF10/iPrOH90); 9c: SCO+PF14+3 mM Ca (PF14 dissolved in THF10/iPrOH90); 10a: SCO+PF14 (PF14 dissolved in EtOH90/DMSO10/PyC0.4); 10b: SCO+PF14+3 mM Mg (PF14 dissolved in EtOH90/DMSO10/PyC0.4); 10c: SCO+PF14+3 mM Ca (PF14 dissolved in EtOH90/DMSO10/PyC0.4). DMF—N,N-dimethylformamide; EtOH—ethanol; iPrOH—isopropanol; THF—tetrahydrofuran; PyC—trimethylene carbonate. Each dataset represents mean±SD of technical replicates from two independent experiments. Data was analyzed by one-way ANOVA with Tukey's test, asterisks indicate statistically significant difference, compared to the same solution from “MQ water” group (i.e. columns 3, 4 and 5), * p-value <0.05, ** p-value <0.005, **** p-value <0.0001.

FIG. 5. Effect on splicing correction by nanoparticles involving cargo nucleotide SCO-705 and two differently dissolved CPPs (PF14 and hPep3).

1 mM stock solutions of PF14 and hPep3 were prepared by dissolving the peptides in different solvents and their mixtures (proportions are given as v:v). Nanoparticles of 100 nM SCO-705, 500 nM peptide and 3 mM MgCl2 or CaCl2 were prepared by mixing the components in MQwater. After 30 min incubation, solutions were diluted 10-fold with cell culture medium and added to HeLa pLuc 705 cells. Luciferase activity was measured after 24 h of incubation. As a negative control, cells were incubated with a medium containing 10% (v:v) of MQwater (untreated cells).

In the graph the numbers on the X-axis represent: 1: Untreated cells; 2: SCO; 3a: SCO+PF14 (PF14 dissolved in water); 3b: SCO+PF14+3 mM Mg (PF14 dissolved in water); 3c: SCO+PF14+3 mM Ca (PF14 dissolved in water); 4a: SCO+PF14 (PF14 dissolved in EtOH90/DMSO10/PyC0.4); 4b: SCO+PF14+3 mM Mg (PF14 dissolved in EtOH90/DMSO10/PyC0.4); 4c: SCO+PF14+3 mM Ca (PF14 dissolved in EtOH90/DMSO10/PyC0.4); 5a: SCO+hPep3 (hPep3 dissolved in water); 5b: SCO+hPep3+3 mM Mg (hPep3 dissolved in water); 5c: SCO+hPep3+3 mM Ca (hPep3 dissolved in water); 6a: SCO+hPep3 (hPep3 dissolved in DMSO); 6b: SCO+hPep3+3 mM Mg (hPep3 dissolved in DMSO); 6c: SCO+hPep3+3 mM Ca (hPep3 dissolved in DMSO); 7a: SCO+hPep3 (hPep3 dissolved in DMSO10/EtOH90); 7b: SCO+hPep3+3 mM Mg (hPep3 dissolved in DMSO10/EtOH90); 7c: SCO+hPep3+3 mM Ca (hPep3 dissolved in DMSO10/EtOH90); 8a: SCO+hPep3 (hPep3 dissolved in DMSO10/iPrOH90); 8b: SCO+hPep3+3 mM Mg (hPep3 dissolved in DMSO10/iPrOH90); 8c: SCO+hPep3+3 mM Ca (hPep3 dissolved in DMSO10/iPrOH90).

EtOH—ethanol; DMSO—dimethyl sulfoxide; PyC—trimethylene carbonate; iPrOH—isopropanol. Each dataset represents mean±SD of technical replicates from three independent experiments. Data was analyzed by one-way ANOVA with Tukey's test, asterisks indicate statistically significant difference between two indicated datasets, ** p-value <0.005, **** p-value <0.0001.

FIG. 6. Effect on luciferase expression by nanoparticles involving siRNA cargo that targets luciferase mRNA and differently dissolved CADY CPP.

1 mM stock solutions of CADY were prepared by dissolving the peptide in different solvents and their mixtures (proportions are given as v:v). Nanoparticles of 15 nM siRNA, 510 nM CADY and 3 mM MgCl2 or CaCl2 were prepared by mixing the components in MQ water. After 30 min incubation, solutions were diluted 10-fold with cell culture medium and added to luciferase-expressing cells, U87 MG-Luc2. Luciferase activity was measured after 48 h of incubation. As a negative control, cells were incubated with siRNA only. As a positive control, siRNA was transfected with Lipofectamine RNAiMAX. In the graph the numbers on the X-axis represent: 1: siRNA only; 2: siRNA+RNAiMAX; 3a: siRNA+CADY (CADY dissolved in water); 3b: siRNA+CADY+3 mM Mg (CADY dissolved in water); 3c: siRNA+CADY+3 mM Ca (CADY dissolved in water); 4a: siRNA+CADY (CADY dissolved in DMSO); 4b: siRNA+CADY+3 mM Mg(CADY dissolved in DMSO); 4c: siRNA+CADY+3 mM Ca (CADY dissolved in DMSO); 5a: siRNA+CADY (CADY dissolved in DMS010/EtOH90); 5b: siRNA+CADY+3 mM Mg(CADY dissolved in DMS010/EtOH90); 5c: siRNA+CADY+3 mM Ca (CADY dissolved in DMS010/EtOH90) 6a: siRNA+CADY (CADY dissolved in DMSO10/iPrOH90); 6b: siRNA+CADY+3 mM Mg(CADY dissolved in DMSO10/iPrOH90); 6c: siRNA+CADY+3 mM Ca (CADY dissolved in DMSO10/iPrOH90).

DMSO—dimethyl sulfoxide; EtOH—ethanol; iPrOH—isopropanol. Each dataset represents mean±SD of three technical replicates. Data was analyzed by one-way ANOVA with Tukey's test, asterisks indicate statistically significant difference between two indicated datasets, * p-value <0.05, ** p-value <0.005, **** p-value <0.0001.

FIGS. 7a and 7b. Effect on luciferase expression by nanoparticles involving mRNA cargo encoding luciferase and differently dissolved CPPs (PF14 and C22-PF14).

1 mM stock solutions of PF14 and C22-PF14 were prepared by dissolving the peptides in different solvents and their mixtures (proportions are given as v:v). Nanoparticles of 200 ng mRNA, 1040 nM peptide and 3 mM MgCl2 or CaCl2 were prepared by mixing the components in MQwater. After 30 min incubation, solutions were added to HaCaT cells. Luciferase activity was measured after 24 h of incubation. As a negative control, cells were incubated with a medium containing 10% (v:v) of MQ water (untreated cells). As a positive control, mRNA was transfected with Lipofectamine MessengerMAX (MMAX). In FIG. 7a is shown Luciferase expression induced by all solutions studied. In FIG. 7b is shown luminescence of cells incubated with particles of mRNA and peptides shown separately. In the graph the numbers on the X-axis represent: 1: Untreated cells; 2: mRNA; 3: mRNA+MMAX; 4a: mRNA+PF14 (PF14 dissolved in ETOH90/DMSO10/PyC0.4); 4b: mRNA+PF14+3 mM Mg (PF14 dissolved in ETOH90/DMSO10/PyC0.4); 4c: mRNA+PF14+3 mM Ca (PF14 dissolved in ETOH90/DMSO10/PyC0.4); 5a: mRNA+C22-PF14 (C22-PF14 dissolved in water); 5b: mRNA+C22-PF14+3 mM Mg (C22-PF14 dissolved in water); 5c: mRNA+C22-PF14+3 mM Ca (C22-PF14 dissolved in water); 6a: mRNA+C22-PF14 (C22-PF14 dissolved in EtOH); 6b: mRNA+C22-PF14+3 mM Mg (C22-PF14 dissolved in EtOH); 6c: mRNA+C22-PF14+3 mM Ca (C22-PF14 dissolved in EtOH) 7a: mRNA+C22-PF14 (C22-PF14 dissolved in EtOH90/DMSO10); 7b: mRNA+C22-PF14+3 mM Mg (C22-PF14 dissolved in EtOH90/DMSO10); 7c: mRNA+C22-PF14+3 mM Ca (C22-PF14 dissolved in EtOH90/DMSO10); 8a: mRNA+C22-PF14 (C22-PF14 dissolved in iPrOH90/DMSO10); 8b: mRNA+C22-PF14+3 mM Mg (C22-PF14 dissolved in iPrOH90/DMSO10); 8c: mRNA+C22-PF14+3 mM Ca (C22-PF14 dissolved in iPrOH90/DMSO10).

EtOH—ethanol; DMSO—dimethyl sulfoxide; PyC—trimethylene carbonate; iPrOH—isopropanol. Each dataset represents mean±SD of three technical replicates. Data was analyzed by one-way ANOVA with Tukey's test, asterisks indicate statistically significant difference between indicated datasets, **** p-value <0.0001.

### DETAILED DESCRIPTION OF THE INVENTION

The present inventors have found that prior art methods for producing CPP-containing nanoparticles leave a substantial amount of CPP peptide remaining in solution as ill-defined aggregates. This leads to low reproducibility of results and have detrimental effects on the experiments in vivo.

To avoid formation of ill-defined aggregates, the present inventors have created protocols for dissolving the CPPs in mixtures of organic solvents. After dispersion of such stock solutions in water, the peptides form uniform and spherical nanoparticles, which upon combination with cargo molecules yield homogeneous particles that are efficiently taken up by cells and lead to high activity of cargo molecules inside living cells.

It has furthermore been realized by the present inventors that the use of trifluoroacetic acid (TFA) in the preparation and purification of cell-penetrating peptides result in various amounts of residual TFA in commercial preparations of CPPs. Sometimes, TFA is also added during preparation of nanoparticles as presence of TFA in an aqueous CPP solution may improve the formation and biological activity of nanoparticles. However, TFA is also toxic and it is thus desirable to reduce TFA content in a composition that is to be administered to live cells or as a pharmaceutical composition for clinical use. The presence of TFA also reduces the shelf-life of compositions comprising CPP nanoparticles. The method according to the invention makes it possible to prepare compositions comprising CPP nanoparticles, which compositions are free of TFA.

The suitable mixtures of solvents contain different types of alcohols and aprotic solvents. The CPP nanoparticles can be assembled on a core of organic cyclic carbonate, or without such a core.

In an exemplary embodiment of the invention, the method for producing nanoparticles comprise the following steps.

The cell-penetrating peptide is dissolved in dry alcohol. Suitable concentrations are in the range 1.5-8 mM.

The order of mixing is usually not important. It is also possible to first dissolve the CPP in the aprotic solvent, and then add the alcohol.

An aprotic solvent, such as pure DMSO, DMF or THF, is added to a concentration of 1-50% (volume/volume), depending on the peptide and the content of counter-ions.

For assembling the CPP micelle on a core, an organic cyclic carbonate is optionally added to 0.1-5% (v/v) concentration.

The concentration of CPP may be adjusted to about 1 mM with the respective mixture of solvents.

CPP solution may be diluted 5-fold to 20-fold into pure water (quick mixing or linear flow mixing microfluidics).

The CPP solution in water (5 to 200 μM, generally 0.1 to 0.01 mM) is then used for assembling nanoparticles with cargo molecules.

Divalent metal ions, preferably Ca²⁺ or Mg²⁺, may be added to CPP-NA nanoparticles at concentrations from 0.01 to 5 mM concentration for increasing the zeta potential of particles and the biological effect of the nucleic acids inside cells.

### Cell-Penetrating Peptides

CPPs that can be used include commercially available CPPs, including but not limited to the ones presented below.

| | | N-terminal | | |
|---|---|---|---|---|
| Peptide | Amino acid sequence | modification | Ref. | SEQ ID NO: |
| PF14 (PepFect 14) | AGYLLGKLLOOLAAAALOOLL | Stearyl (C18) | (3) (4) | 1 |
| PF14-C22 | AGYLLGKLLOOLAAAALOOLL | Behenyl (C22) | (5) | 2 |
| PF6 (PepFect 6) | AGYLLGK^{a}INLKALAALAKKIL | Stearyl (C18) | (6) | 3 |
| PF14-OA | AGYLLGKLLOOLAAAALOOLL | Oleyl (C18:1) | | 4 |
| PF14-IAH | AGYLLGKLLAOLAOOALOALL | Stearyl (C18) | | 5 |
| PF14-4H | HHYHHGKLLOOLAAAALOOLL | Stearyl (C18) | | 6 |
| PF14-6H | HHYHHYHHGKLLOOLAAAALOOLL | Arachidyl (C20) | | 7 |
| NF51 (NickFect 51) | AGYLLGO^{b}INLKALAALAKKIL | Stearyl (C18) | (7) | 8 |
| NF55 (NickFect 55) | AGYLLGO^{b}INLKALAALAKAIL | Stearyl (C18) | (8) | 9 |
| NF70 (NickFect 70) | HHHHYHHGO^{b}ILLKALKALAKAIL | Arachidyl (C20) | (9) | 10 |
| NF71 (NickFect 71) | HHYHHGO^{b}ILLKALKALAKAIL | Stearyl (C18) | (9) | 11 |
| NF72 (NickFect 72) | HHHHHHYHHGO^{b}ILLKALKALAKAIL | Arachidyl (C20) | (9) | 12 |
| WRAP1 | LLWRLWRLLWRLWRLL | — | (10) | 13 |
| WRAP5 | LLRLLRWWWRLLRLL | — | (9) | 14 |
| CADY | GLWRALWRLLRSLWRLLWRA^{c} | Acetyl (C2) | 21 | 15 |
| hPep3 | L(R8)^{d}KLAKA(R8)AKLLKA(R8)AKAL | — | 22 | 16 |

^{a}To the side chain of lysine a lysine tree of three Lys residues is coupled to which 4 trifluoroquinoline molecules are attached
^{b}the glycine residue is coupled to the side chain of ornithine (δ-amino group) instead of α-amino group
^{c}C-terminus of peptide is cysteamide
^{d}(R8) = (R)-2-(7-octenyl)alanine

The C-terminus of all used peptides (except CADY) is amide.

### Alcohols

Alcohols useful as solvents in the present invention include methanol, ethanol, 1-propanol, 1-butanol, 1-pentanol, 1-hexanol, 2-propanol, 2-butanol, 2-pentanol, 2-hexanol, tert-butanol, and 2-ethyl 1-hexanol.

The alcohol may be chosen based on the fatty acid modification of the CPP. The longer the fatty acid molecule, the longer the alcohol chain should be.

### Aprotic Solvents

Aprotic solvents useful in the present invention include, but are not limited to dimethyl sulfoxide, dimethyl formamide and tetrahydrofuran. The preferred aprotic solvent is dimethyl sulfoxide that FDA assigns to Class 3 of organic solvents together with ethanol and propanol that are considered the least toxic and of lowest risk to human health. The aprotic solvent is typically added to the dry alcohol to a concentration of at least 1% up to 50% (volume/volume), depending on the peptide and the content of counter-ions. The aprotic solvent may be added to a concentration of at least 2%, 3%, 4%, 5%, 10%, 20%, 30%, or 40%, or up to a concentration of 10%, 20%, 30%, or 40%, or 50% (volume/volume).

### Quality Control

The diameter and zeta potential of CPP nanoparticles may be measured using dynamic light scattering (DLS) or nanoparticle tracking analysis system (NTA) (1), (4) (using 0.01 mM peptide). The morphology of NP is analysed by transmission electron microscopy (TEM) (11), scanning electron microscopy (SEM) or atomic force microscopy (AFM) using 0.1 or 0.01 mM peptide solution.

### Cargo Molecules

Suitable cargo molecules for use in the present invention are negatively charged molecules, such as negatively charged peptides and proteins, and nucleic acids. Nucleic acids include both DNA and RNA molecules of any length, such as mRNA, siRNA, miRNA, antisense oligonucleotide, splicing correcting oligonucleotide, plasmid DNA etc.

### EXPERIMENTAL

### Example 1

The following cell-penetrating peptides (CPPs) were used: PF14, PF14-IAH, PF14-OA, PF14-4H, PF14-6H, C22-PF14, NF51, NF55, PF6, NF70, NF71, NF72, WRAP1, WRAP5, CADY, and hPep3 The peptides were purchased from PepScan (Netherlands), Pepmic (China), GL Biochem (China), or were synthesized in-house at the Institute of Technology, University of Tartu. Before the experiments the known amount (measured by the peptide-synthesis company, or estimated by the weight of the lyophilised peptide) of peptides were dissolved at 1-2 mM concentration in Milli-Q quality water or, if necessary, in 1 mM solution of trifluoroacteic acid (TFA) in Milli-Q water, according to the prior art, or with organic solvents according to the invention. Between experiments the peptide stock solutions were stored at −20° C.

The physical characteristics (diameter and zeta-potential) of the peptide micelles/aggregates/nanoparticles were analysed by dynamic light scattering (DLS) using a ZetaView (Particle Metrix, Germany) or ZetasizerNano ZS apparatus (Malvern Instruments, UK) at 10 μM concentration in water.

DLS analysis of the size of CPP particles forming after 100-fold dilution of 1 mM peptide solution in Milli-Q water or organic solvents (ambient temperature) is provided in Table 1.

TABLE 1

| Stock solution | Diameter (nm) | SD | PDI |
|---|---|---|---|
| PF14 in MilliQ | 317.9 | 114.2 | 0.563 |
| PF14 in DMSO | 300.5 | 70.0 | 0.578 |
| PF14 in MeOH | 310.9 | 76.1 | 0.522 |
| PF14 in DMSO/MeOH (1/1) | 260.3 | 51.0 | 0.579 |
| PF14 in PrOH | 156.5 | 19.4 | 4.19 |
| PF14 in DMSO/iPrOH (1/1) | 95.9 | 1.0 | 0.117 |

The optimal size of nanoparticles to be used for drug delivery in vivo is in range of 100 nm (between 10 and 200 nm), and the lack of large aggregates is highly essential. In order to analyse the size of CPP clusters that form in water, DLS traces were measured by Malvern Zetasizer Nano ZS. Four consecutive measurements were performed with each sample for the calculation of the size and polydispersity of the forming CPP particles.

Organic solvents that are commonly used for dissolving peptides, like DMSO and methanol, solubilised lyophilised PF14 quickly and peptide micelles that formed after dilution of concentrated solution in water were of similar size with particles forming with water-dissolved PF14. However, the particles that formed from PF14 dissolved in organic solvents upon dilution, showed less wide distribution of sizes compared to peptide dissolved in water. (Table 1)

PF14 solutions in alcohols with longer aliphatic chain than methanol, like ethanol and propanol, yielded upon dilution in water smaller and more homogeneous particles compared to concentrated solution of PF14 dissolved in water. Compared to solutions made in alcohol, their mixtures with DMSO were beneficial regarding both the size and homogeneity of forming particles. (Table 1)

Among alcohols with different number of carbon atoms and branching of the aliphatic chain, most preferred are ethanol, 2-propanol and 2-butanol for dissolving PF14, whose solutions yield smaller and more homogeneous particles upon dilution into water when compared to methanol and alcohols with more carbon atoms or branching of aliphatic chain. (Tables 2 and 3)

TABLE 2
Particle size (in nanometer, nm) of PF14 forming from 1 mM solution
in different alcohols and their mixtures with organic solvents
upon 100-fold (first 10-fold dilution, and then further 10-fold)
dilution in water to a final concentration of 10 μM.

| SIZE | Mean | SD | Pdl |
|---|---|---|---|
| PF14 in PrOH | 275.3 | 49.4 | 0.628 |
| PF14 in iPrOH | 156.5 | 19.4 | 0.419 |
| PF14 in BuOH | 334.7 | 81.8 | 0.477 |
| PF14 in nAmOH | 457.4 | 19.0 | 0.518 |
| PF14 in secBuOH | 103.7 | 22.9 | 0.493 |
| PF14 in tBuOH | 264.2 | 94.4 | 0.488 |
| PF14 in iAmOH | 431.7 | 64.1 | 0.607 |
| PF14 in secAmOH | 293.1 | 22.9 | 0.518 |
| PF14 in2-Ethyl 1-hexanol | 798.7 | 176.5 | 0.645 |
| PF14 in PC(10%)/MeOH(90%) | 272.2 | 74.6 | 0.533 |
| PF14 in iPrOH/DMSO | 113 | 1.4 | 0.093 |
| PF14 in MQ | 426.2 | 44.9 | 0.501 |

MQ—Milli-Qwater, PC— propylene carbonate.

TABLE 3
Mixtures of DMSO with ethanol or isopropanol
and cyclic organic carbonates:

| | Mean size | Pdl | SD |
|---|---|---|---|
| PF14 in EtOH | 101.6 | 0.213 | 4.13 |
| PF14 in EtOH90/DMSO10 | 93.18 | 0.259 | 4.20 |
| PF14 in EtOH90/DMSO5/PyC5 | 154.4 | 0.107 | 1.59 |
| PF14 in EtOH90/DMSO5/EC5 | 105.8 | 0.329 | 10.34 |
| PF14 in iPrOH | 115.2 | 0.301 | 15.83 |
| PF14 in iPrOH90/DMSO10 | 78.96 | 0.246 | 11.46 |
| PF14 in iPrOH90/DMSO5/PyC5 | 127.9 | 0.112 | 1.34 |
| Standard 100 nm | 109.8 | 0.006 | 1.25 |

Supplementation of PF14 solution in alcohol with optimal concentration (1-50%) of DMSO decreases the size and polydispersity of peptide particles forming upon dilution in water. Addition of cyclic organic carbonates (e.g. ethylene carbonate, EC, or trimethylene carbonate, PyC) into PF14 solution in alcohol/DMSO mixture, increases the size and homogeneity of forming peptide particles. (Table 3). The formation of more homogeneous particles could be caused by formation of core of peptide particles by cyclic carbonate that has limited solubility in water. 100 nm standard—Nanosphere Size Standards, 100 nm, Thermo Scientific.

TABLE 4
Optimal compositions of PF14 solutions containing ethanol, DMSO
and trimethylene carbonate. Reduction of DMSO and trimethylene
carbonate concentration in solution of PF14 in ethanol reduces
the size of peptide particles and their polydispersity.

| | Mean size | SD | Pdl |
|---|---|---|---|
| PF14 in EtOH | 87.2 | 0.8 | 0.283 |
| PF14 in EtOH90/DMSO10 | 158.1 | 3.1 | 0.275 |
| PF14 in EtOH90/DMSO9/PyC1 | 115.6 | 1.8 | 0.107 |
| PF14 in EtOH98/DMSO1/PyC1 | 94.3 | 2.9 | 0.217 |
| EtOH99/DMSO0.25/PyC0.25 | 159.3 | 5.2 | 0.198 |
| Standard 100 nm | 114.2 | 1.7 | 0.018 |

TABLE 5
The optimal solvent composition for dissolving PF14
is not optimal for other CPPs of PF and NF series,
and has to be optimised for each peptide.

| | Mean size | SD | Pdl |
|---|---|---|---|
| PF14-OA iPrOH90/DMSO10 | 217.4 | 72.4 | 0.664 |
| PF14-OA in iPrOH75/DMSO25 | 65.8 | 10.5 | 0.355 |
| C22-PF14 in iPrOH90/DMSO10 | 143.4 | 66.7 | 0.559 |
| C22-PF14 in iPrOH90 | 123.0 | 22.3 | 0.348 |
| NF70 in iPrOH78/DMSO20/PyC2 | 161.4 | 1.9 | nd |
| NF70 in EtOH78/DMSO20/PyC2 | 143.9 | 3.0 | nd |
| NF71 in iPrOH78/DMSO20/PyC2 | 144.1 | 1.3 | nd |
| NF71 in EtOH78/DMSO20/PyC2 | 181.1 | 3.7 | nd |

Analysis of the size and shape of CPP nanoparticles and their complexes with cargo nucleic acids by transmission electron microscopy (TEM) confirmed data from DLS analysis. Depending on the solvent used for preparation of 1 mM peptide solutions, the particles of CPP were largely different.

PF14 of high quality (e.g. prepared by Pepscan or Caslo) does not contain residual TFA from the purification of peptide. Dissolution of such PF14 in Milli-Q water at 1 mM yields filamentous micelles and aggregates, whose size varies from 200 nm to more than 1 μm (Solution 1). Dilution of obtained solution in water does not substantially dissociate micelles into small peptide nanoparticles, and the complexes with nucleic acid are not always active in cells (e.g. in FIGS. 7a and 7b).

Dissolution of PF14 at 1 mM concentration in weakly acidic water (1 mM, equimolar TFA in MilliQ water) leads to formation of markedly smaller micelles and aggregates, in average with 100-500 nm diameter (Solution 2). Dilution of this solution ten-fold in Milli-Q water and formation of complex with nucleic acid cargo yields nanoparticles that are endocytosed by cells and the nucleic acid exhibits its activity (siRNA, SCO etc.). In parallel, the larger micelles/aggregates are also present in the 1 mM solution of peptide, but this fraction is relatively small.

Different CPPs dissolved in TFA-containing water (Solutions 2 of CPPs) condense nucleic acid cargo to nanoparticles that have the most uniform size and shape with plasmid DNA, and the most variable with siRNA. The CPP/SCO and CPP/miRNA nanoparticles have the homogeneity between these two (11).

In addition to uniform nanoparticles formed by Solution 2 of CPPs with nucleic acid molecules, large aggregates are always present in the mixtures at lower or higher extent. These could be formed due to the presence of aggregates in the CPP Solution 1 and 2 that are not dissociated upon complex formation with nucleic acid molecules. Such aggregates are not present when CPPs are dissolved first in a mixture of organic solvents at 1 mM concentration, and then diluted in water tenfold.

When the CPP dissolved in water (Solution 2) is complexed with high concentration of NA (siRNA) cargo that is usually required for in vivo applications in animal models, mostly the large aggregates form. The administration of aggregated nanoparticles to an experimental animal is often associated with serious side effects.

CPP dissolved in TFA-containing water (Solution 2 of PF14) condenses pDNA to a mixture of nanoparticles and aggregates that are present in specimen at the same time. The proportion of aggregated material fraction can be reduced by reducing the concentration of CPP and nucleic acid used for preparation of nanoparticles or filtration as demonstrated by Freimann et al. (8) However, both these approaches are time consuming and filtration causes remarkable losses of material.

Dissolution of CPPs of PF and NF series in the mixture of organic solvents of optimal composition, and dilution into water leads to formation of uniform, spherical nanoparticles of peptides. Nanoparticles of PF14 obtained by dissolution of peptide in the mixture of isopropanol, DMSO and trimethylene carbonate (90/9/1) at 1 mM concentration, and quick dilution in water to 0.1 mM concentration are highly regular spheres with ^{˜}40-60 nm diameter as visualised by transmission electron microscopy. No aggregates are present in specimen. DLS data suggest that larger PF14 particles ^{˜}100-150 nm that are observed in TEM images, dissociate upon (further) 10-fold dilution to give population of uniform NP with average diameter ^{˜}100 nm.

### Formation of Nanoparticles/Complexes

### Preparation of Nanoparticles in Water

PF14 was mixed with SCOs (sequence CCU CUU ACC UCA GUU ACA, SEQ ID NO: 17) at a 5:1 molar ratio (MR) in MQ-water, using concentrations of 1 μM of SCO and 5 μM of PF14 (3). Complexes were formed for 30 minutes at room temperature. For preparation of nanoparticles containing Ca or Mg ions, SCO-PF14 complexes were formed by 15 min incubation at room temperature. CaCl₂ or MgCl₂ solutions were then added, and the solutions were mixed by pipetting up and down or on a vortex unit. After 15 min, the solutions were diluted 10-fold with growth medium pre-warmed to 37° C., to reach the final volume for application to the cells. SCO was applied at a final concentration of 100 nM, and Ca and Mg ions at 0 to 5 mM concentration. The optimal molar ratio for CPP to SCO is in the range of 3 to 10, and depends on the charge of used CPP and the used cell type. The addition of divalent cations into PF14/SCO nanoparticles has the highest effect at MR5.

The nanoparticles of CPP with pDNA are prepared in analogous manner. In the most preferred conditions pDNA (pLuc2 or pGL3) at concentration 10 μg/ml is complexed with 10 μM PF14 in Milli-Q water. The complexes are formed at ambient temperature for 30 min, and if required Ca or Mg ions are incorporated in CPP-NA nanoparticles as described above. At the used concentration the N/P ratio for peptide to nucleic acid is 2 (N meaning number of amino groups in the peptide side chain and P number of phosphate groups in the nucleic acid molecule) (also called charge ratio). Depending on the charge of used CPP, type of cells and experiment, N/P ratio can be varied in the range 1-8.

The CPP nanoparticles with miRNA are formed in analogous manner with SCO and pDNA (2). The preferred molar ratio for CPP to siRNA is 17 that corresponds to N/P ratio 2 for PF14 and other CPPs with calculated charge +5. Depending on the charge of CPP, type of cells and experiment, and concentration of miRNA, MR in range 12-35 are applied. If required Ca or Mg ions are incorporated in CPP-miRNA nanoparticles as described above. For delivery of miRNA into primary cells, preferentially Mg ions are incorporated in CPP nanoparticles.

The CPP nanoparticles with siRNA are formed in analogous manner with SCO and pDNA (9). The preferred N/P ratio is 2 for PF14. Depending on the charge of CPP, type of cells and experiment, and concentration of miRNA, MR in range 10-40 are applied. If required, Ca or Mg ions are incorporated in CPP-siRNA nanoparticles as described above. For delivery of siRNA into primary cells, preferentially Mg ions are incorporated in CPP nanoparticles. Cells are incubated with CPP-siRNA nanoparticles for 48 h.

The CPP nanoparticles with mRNA are formed in a similar manner with pDNA. mRNA at concentration 10 μg/ml is complexed with 10 μM PF14 CPP in Milli-Qwater. If required, Ca or Mg ions are incorporated in CPP-mRNA nanoparticles as described above. Unlike other particle solutions described above, CPP-mRNA 10× solutions are directly added into growth medium where cells have been incubated. The preferred N/P ratio (“charge ratio”) of CPP to mRNA is 2. Depending on the charge of used CPP, type of cells and experiment, N/P ratio can be varied in the range 1.5 to 4. For delivery of mRNA into human primary cells, preferentially Mg ions are incorporated in CPP nanoparticles.

### Preparation of Nanoparticles Using CPPs Dissolved in Organic Solvents

The 1 mM solution of CPP prepared in the organic solvent or in their mixture is diluted in Milli-Q water to 0.1 mM solution. The complexes/nanoparticles of CPP with nucleic acid molecules are formed by quick mixing preferentially in Milli-Q water as described above for assembly of complexes with CPPs dissolved in water. The CPP-NA complexes can be assembled also in buffer solution (11) and solutions containing glucose (12). The preferred buffer solutions are HEPES (11) and phosphate (5) buffers.

Delivery of Nucleic Acid Cargo into Cells

The efficiency of the peptide nanoparticles to deliver nucleic acid cargo was analysed in luciferase reporter cell line HeLa pLuc705 cells as previously described (11).

This assay builds on HeLa cells having stably integrated into their genome a luciferase gene with an introduced aberrant splicing site (from beta-globin). Reporter cells (HeLa pLuc 705) thus express protein which has no luciferase activity, due to incorrect splicing. When oligonucleotide that binds to aberrant splicing site (splicing correcting oligo, SCO) is transduced into cells (into nucleus finally), it blocks wrong splicing and active luciferase is synthesised in cells. The activity of luciferase is easy to measure by luminescence with high throughput. This is a very sensitive and specific assay to measure delivery of oligonucleotides into cells.

The EGFP assay in HeLa-EGFP cells works with the same principle. The aberrant splicing site has different nucleotide sequence as well as the oligonucleotide used for masking it, but the SCO binds to pre-mRNA, blocks wrong splicing and leads to expression of fluorescent protein that can be measured by FACS or analysed by fluorescence microscopy (20).

24 hours prior to the experiments, 7000, 10 000 or 20 000 HeLa pLuc705 cells per well were seeded into 96-well plates.

PF14 complexes with SCOs at 5:1 molar ratio were diluted in media used for culturing cells. The final concentrations were 100 nM SCO and 500 nM of PF14 per well. The cells were incubated with the complexes for 24 hours, and then lysed using cell-culture lysis reagent (Promega, Sweden) for 15 min in ice. The luciferase activity was measured using Promega's luciferase assay system on a GLOMAX™ 96 microplate luminometer (Promega, Sweden), and normalized to the protein content which was determined using the Lowry method (BioRad, USA).

For quantification of the oligonucleotide cargo delivery by FACS or fluorescence microscopy, the reporter cell line HeLa EGFP 654 and the corresponding SCO 654 (GCU AUU ACC UAA ACC CAG, SEQ ID NO: 16) were used in analogous settings. Cells were incubated with 100 nM SCO 654 complexed with 500 nM of PF14 for 24 h, detached from plastic, an expression of EGFP was analysed by flow cytometry (BD FACSAria Cell Sorter, BD Biosciences, USA) using the respective analysis software (BD Biosciences, Germany).

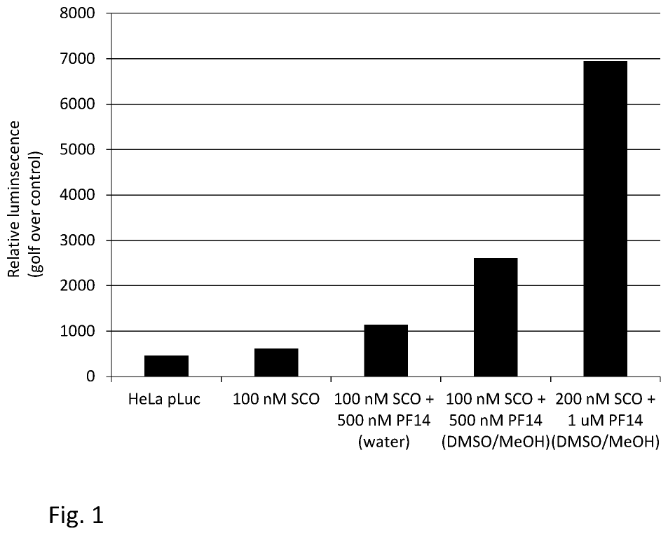

PF14 dissolved in the mixture of organic solvents led to higher biological responses, compared to PF14 stocks prepared in water as shown in FIG. 1. The luminescence signal is significantly increased for complexes of SCO and PF14 when prepared in organic solvent according to the invention, as compared to complexes prepared in water. Also when the cargo consisted of siRNA, miRNA, and pDNA, as shown by the respective assays in living cells (2) (12).

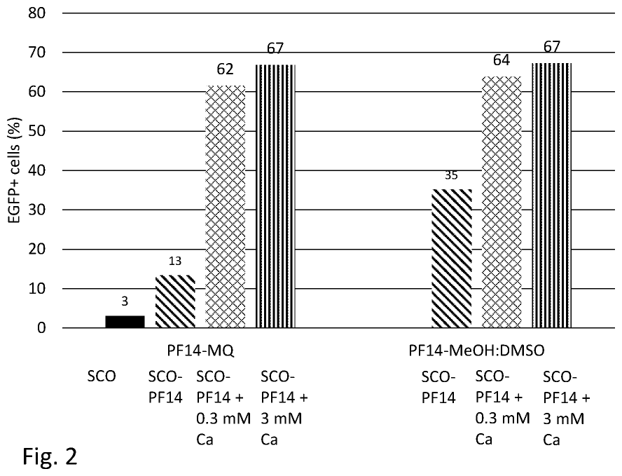

The SCO 654 complexes with PF14 (that was initially dissolved in the mixture of organic solvents and then diluted with water) were also more efficient in splicing redirection in HeLa EGFP 654 cells than PF14 directly dissolved in water (FIG. 2). Addition of the naked SCO 654 into culture medium over the cells induced expression of EGFP in about 2% of cells, and the nanoparticles consisting of SCO and PF14 dissolved in water increased the fraction of cells expressing EGFP to 13%. The highest splicing correction with SCO complexes assembled with PF14 that was dissolved according to the invention, which led to expression of EGFP in 35% of cells in population (FIG. 2). Inclusion of Ca ions into these nanoparticles increased the proportion of EGFP-expression cells to 64% at 0.3 mM, and 67% at 3 mM concentration, respectively.

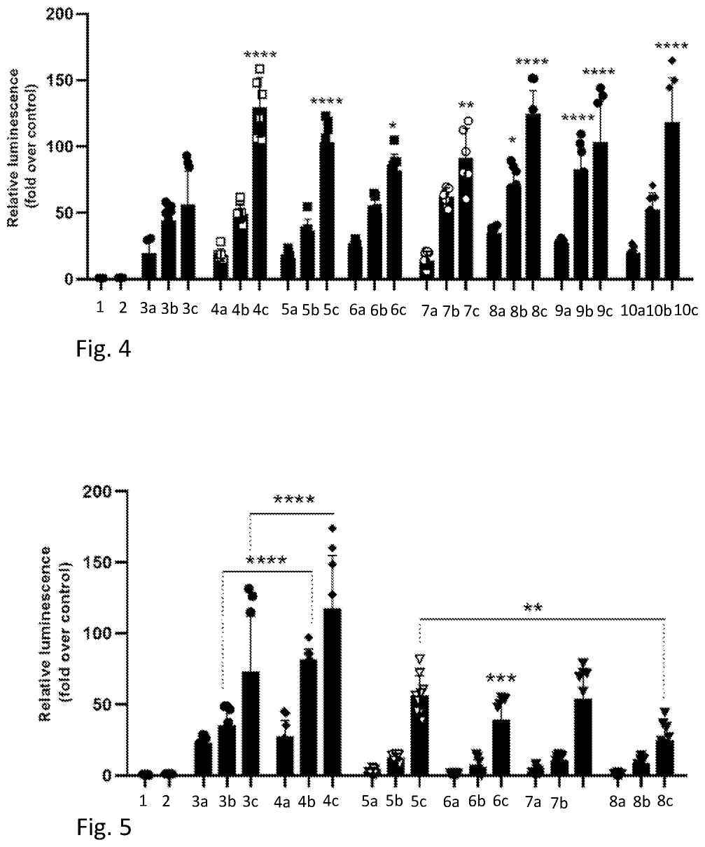

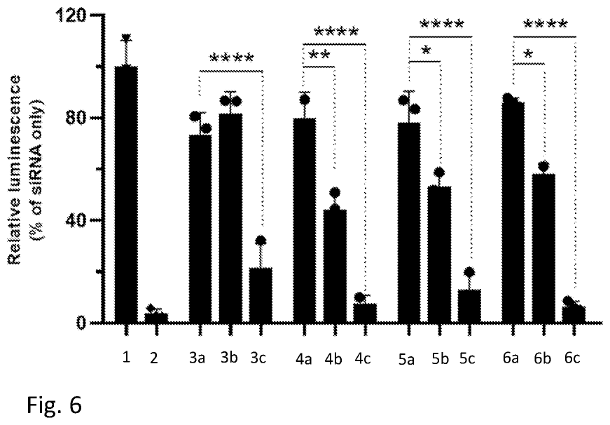

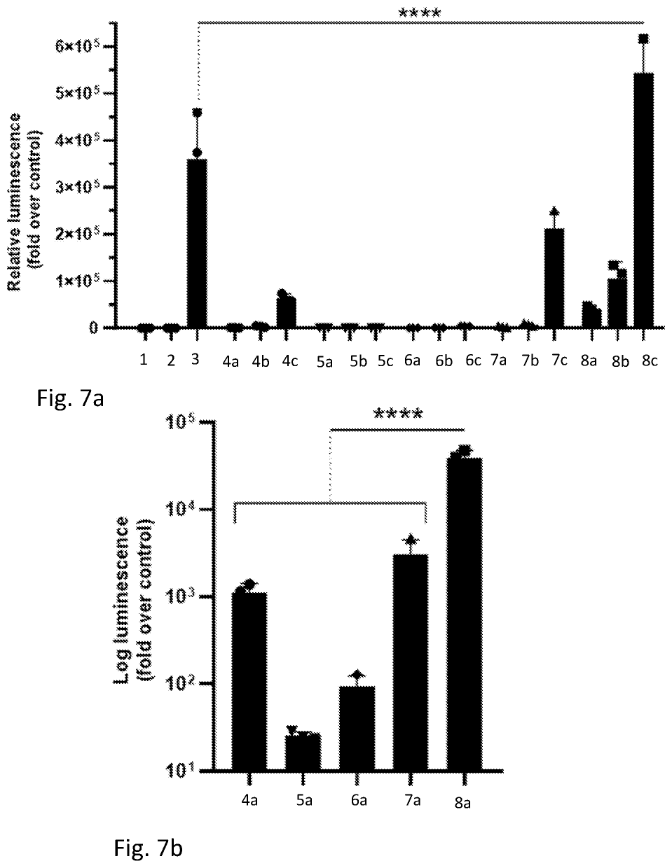

Although dimethyl sulfoxide (DMSO) is the preferred aprotic solvent for dissolving membranophilic CPPs along with selected alcohols due to the highest compatibility with experiments in vivo, other aprotic solvents are applicable for dissolving PF14 and other membranophilic CPPs and further preparation of nanoparticles with negatively charged cargo. PF14 dissolved in dimethyl formamide or tetrahydrofuran in combination with ethanol or 2-propanol (isopropanol) guarantees formation of nanoparticles with SCO, which more efficiently redirect splicing in reporter HeLa pLuc 705 cells compared to water-solubilised CPP (FIG. 4). In FIG. 4 is shown a comparison of the SCO cellular delivery efficiency by PF14 CPP dissolved in different solvents. Luminescence signal of reporter HeLa pLuc705 cells treated with splicing correcting 100 nM oligonucleotide (SCO-705) or its complexes with 500 nM PF14 for 24 h. PF14 was dissolved at 1 mM concentration in Milli-Q quality water or a mixture of organic solvents (DMF/alcohol, or THF/alcohol 1/9, v/v). The correction of aberrant splicing by SCO increased luciferase activity (Kole's assay) that was quantified by luminescence intensity (relative luminescence units). As positive control, the effect of SCO in complexes with PF14 dissolved in DMSO/alcohol mixture 1/9 v/v with addition of trimethylene carbonate to 0.4%, was used.

The strategy to dissolve membranophilic CPPs in the mixture of organic solvents instead of water, is advantageous for various membranophilic CPPs, in addition to PepFects and NickFects. hPep3 is a CPP that is more hydrophobic than PFs., hPep3 dissolved in the mixture of dimethyl sulfoxide yields, together with SCO cargo, nanoparticles of the same efficiency as with water-dissolved hPep3 (FIG. 5).

CADY peptide is not applicable for delivering SCO cargo into mammalian cells (not shown here). CADY was therefore dissolved in different solvents and the solutions were used for assembling nanoparticles with siRNA cargo. When supplemented with Ca ions, nanoparticles prepared with water-dissolved CADY were less efficient in suppressing the expression of targeted gene in U87 cells than nanoparticles prepared with solution of peptide dissolved in the mixture of organic solvents (FIG. 6). Remarkably, Mg ions increased the gene knock-down effect of siRNA cargo only in case of nanoparticles prepared with CADY dissolved in the mixture of organic solvents, but not for water-dissolved CADY.

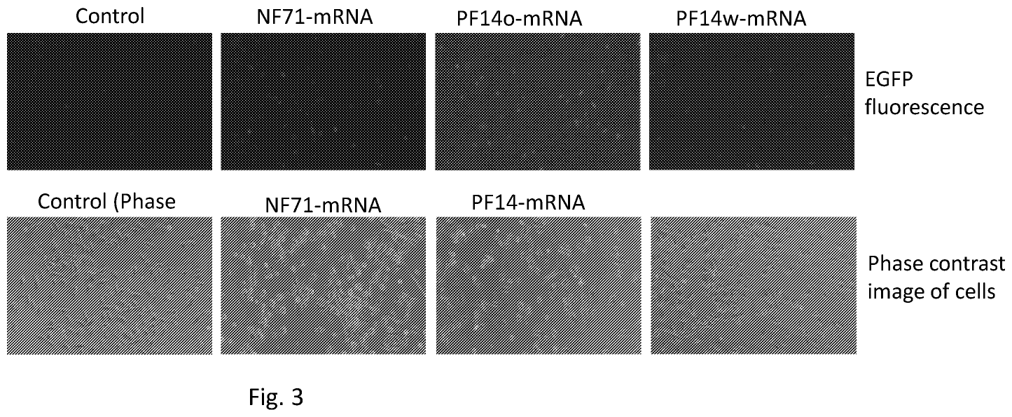

NF71 and PF14 solutions in the mixture of organic solvents that yield optimal CPP nanoparticles upon quick dilution into Milli-Q water, enable transfection of the EGFP mRNA into primary keratinocytes and expression of EGFP after formation of the respective nanocomplexes (FIG. 3). PF14 dissolved in the mixture of organic solvents forms with mRNA nanoparticles that trigger expression of EGFP in higher number of keratinocytes than PF14 dissolved in water (FIG. 3).

More significant difference in the potency to deliver mRNA was detected in case of differently dissolved PF14-C22. PF14-C22 dissolved in mixture of DMSO and isopropanol induced more than 100-fold higher expression of protein from its mRNA than water-dissolved peptide in HaCaT cells that are considered very difficult to transfect (FIGS. 7a and 7b). Importantly, supplementation of mRNA/PF14-C22 nanoparticles with Ca ions enables higher expression of the protein of interest than the best commercially available transfection reagent for mRNA: Lipofectamine MessengerMax (FIG. 7a).

### REFERENCES

1. Status update in the use of cell-penetrating peptides for the delivery of macromolecular therapeutics. Kurikoff et al. 3, 2021, Expert Opin Biol Ther, Vol. 21, pp. 361-370.
2. NickFect type of cell-penetrating peptides present enhanced efficiency for microRNA-146a delivery into dendritic cells and during skin inflammation. Carreras-Badosa et al. 2020, Biomaterials, Vol. 262, p. 120316.
3. Scavenger receptor-mediated uptake of cell-penetrating peptide nanocomplexes with oligonucleotides. Ezzat et al. 3, 2012, FASEB J, Vol. 26, pp. 1172-1180.
4. Solid formulation of cell-penetrating peptide nanocomplexes with siRNA and their stability in simulated gastric conditions. Ezzat et al. 1, 2012, J Control Release, Vol. 162, pp. 1-8.
5. Saturated Fatty Acid Analogues of Cell-Penetrating Peptide PepFect14: Role of Fatty Acid Modification in Complexation and Delivery of Splice-Correcting Oligonucleotides. Lehto et al. 3, 2017, Bioconjug Chem, Vol. 28, pp. 782-792.
6. Design of a peptide-based vector, PepFect6, for efficient delivery of siRNA in cell culture and systemically in vivo. Andaloussi et al. 9, 2011, Nucleic Acids Res, Vol. 39, pp. 3972-3987.
7. PepFects and NickFects for the Intracellular Delivery of Nucleic Acids. Arukuusk et al. 2015, Methods Mol Biol, Vol. 1324, pp. 303-315.
8. Formulation of Stable and Homogeneous Cell-Penetrating Peptide NF55 Nanoparticles for Efficient Gene Delivery In Vivo. Freimann et al. 2018, Mol Ther Nucleic Acids, Vol. 10, pp. 28-35.
9. Enhancement of siRNA transfection by the optimization of fatty acid length and histidine content in the CPP. Porosk et al. 10, 2019, Biomater Sci, Vol. 7, pp. 4363-4374.
10. Deciphering the internalization mechanism of WRAP:siRNA nanoparticles. Deshayes et al. 6, 2020, Biochim Biophys Acta Biomembr, Vol. 1862, p. 183252.
11. Characterization of Peptide-Oligonucleotide Complexes Using Electron Microscopy, Dynamic Light Scattering, and Protease Resistance Assay. Padari et al. 2019, Methods Mol Biol, Vol. 2036, pp. 127-139.
12. Characteristics of Cell-Penetrating Peptide/Nucleic Acid Nanoparticles. Margus et al. 1, 2016, Mol Pharm, Vol. 13, pp. 172-179.
13. Supramolecular Assembly of Aminoethylene-Lipopeptide PMO Conjugates into RNA Splice-Switching Nanomicelles. Kuhn et al. 48, 2019, Advanced Functional Materials, Vol. 29.
14. Pre-administration of PepFect6-microRNA-146a nanocomplexes inhibits inflammatory responses in keratinocytes and in a mouse model of irritant contact dermatitis. Urgard et al. 2016, J Control Release, Vol. 235, pp. 195-204.
15. Up-regulation of luciferase gene expression with antisense oligonucleotides: implications and applications in functional assay development. Kang et al. 18, 1998, Biochemistry, Vol. 37, pp. 6235-6239.
16. Targeted gene silencing in human embryonic stem cells using cell-penetrating peptide PepFect 14. Ervin et al. 1, 2019, Stem Cell Res Ther, Vol. 10, p. 43.
17. Thermodynamic studies and binding mechanisms of cell-penetrating peptides with lipids and glycosaminoglycans. Ziegler, A, 2008, ADVANCED DRUG DELIVERY REVIEWS 60, (4-5), 580-597, DOI0.1016/j.addr.2007.10.005
18. Classes of Cell-Penetrating Peptides, Pooga; M., and Langel, Ü., 2015, Methods Mol Biol. 1324, 3-28.
19. Cell-Penetrating Peptides, Langel; Ü CPP, © Springer Nature Singapore Pte Ltd. 2019, https://doi.org/10.1007/978-981-13-8747-0
20. Divalent metal cations boost cellular delivery of antisense oligonucleotides by cell penetrating peptides. Maloverjan, M.; Padari, K.; Abroi, A.; Rebane, A.; Pooga, M. 2022, Cells 11(4), 756. doi: 10.3390/cells11040756.
21. A new potent secondary amphipathic cell-penetrating peptide for siRNA delivery into mammalian cells. Crombez L, Aldrian-Herrada G, Konate K, Nguyen Q N, McMaster G K, Brasseur R, Heitz F, Divita G., 2009, Mol Ther. 17(1):95-103. doi: 10.1038/mt.2008.215.
22. Novel Orthogonally Hydrocarbon-Modified Cell-Penetrating Peptide Nanoparticles Mediate Efficient Delivery of Splice-Switching Antisense Oligonucleotides In Vitro and In Vivo. Bazaz S, Lehto T, Tops R, Gissberg O, Gupta D, Bestas B, Bost J, Wiklander O P B, Sork H, Zaghloul E M, Mamand D R, Hällbrink M, Sillard R, Saher O, Ezzat K, Smith C I E, Andaloussi S E, Lehto T., 2021, Biomedicines. 9(8):1046. doi: 10.3390/biomedicines9081046.

1. A method of preparing a nanoparticle comprising at least one cell-penetrating peptide (CPP) and a cargo molecule (CM), said method comprising the steps:
Preparing a CPP solution comprising a CPP, a dry alcohol; and 1-50% (v/v) of an aprotic solvent; and
Optionally diluting the CPP solution in water to a concentration of 2 to 500 μM,
Adding the cargo molecule to the CPP solution to obtain a CPP:CM mixture;
whereby nanoparticles comprising CPP and cargo molecule are formed.

2. The method according to claim 1, wherein the aprotic solvent is selected from dimethyl sulfoxide, dimethyl formamide, and tetrahydrofuran.

3. The method of claim 1, wherein the CPP is a membranophilic CPP selected from a CPP having 70-100% hydrophobic amino acids, an amphipathic CPP having 45-75% hydrophobic amino acids, a CPP with an added C10-C24 fatty acid, a CPP with an added fluorinated C10-C24 fatty acid, and/or a CPP with an added cholesterol.

4. The method according to claim 1, wherein the CPP solution further comprises 0.1-5% (v/v) of an organic cyclic carbonate.

5. The method according to claim 4, wherein the organic cyclic carbonate is selected from the group consisting of ethylene carbonate; propylene carbonate; ethyl methyl carbonate (EMC), glycerol carbonate (4-(Hydroxymethyl)-1,3-dioxolan-2-one), and trimethylene carbonate (1,3-dioxan-2-one).

6. The method according to claim 1, wherein the cargo molecule is a negatively charged molecule.

7. The method according to claim 1, further comprising adding a divalent metal ion to the CPP solution or the CPP:CM mixture or to the nanoparticles.

8. The method according to claim 7, wherein the divalent metal ion is Ca²⁺ or Mg²⁺.

9. The method according to claim 1, wherein the dry alcohol is selected from the group consisting of methanol, ethanol, 1-propanol, 1-butanol, 1-pentanol, 1-hexanol, 2-propanol, 2-butanol, 2-pentanol, 2-hexanol, tert-butanol, and 2-ethyl 1-hexanol.

10. The method according to claim 1, wherein the dry alcohol is a branched chain alcohol.

11. The method according to claim 1, wherein the ratio CPP:CM in the CPP:CM mixture is in the range 10:1 to 1:1, preferably 5:1.

12. A nanoparticle comprising a cell-penetrating peptide and a cargo molecule, said nanoparticle being obtainable by a method according to claim 1.

13. A pharmaceutical composition comprising a nanoparticle obtained with the method according to claim 1.


SEQUENCE LISTING (18 sequences)

SEQ ID NO: 1 - protein, 21 aa, Artificial Sequence
  PF14
  MISC_FEATURE (1)..(1): Stearyl (C18)
  MISC_FEATURE (10)..(11): Pyrrolysine
  MISC_FEATURE (18)..(19): Pyrrolysine
AGYLLGKLLXXLAAAALXXLL

SEQ ID NO: 2 - protein, 21 aa, Artificial Sequence
  PF14-C22
  MISC_FEATURE (1)..(1): N-terminal modification Behenyl (C22)
  MISC_FEATURE (10)..(11): Pyrrolysine
  MISC_FEATURE (18)..(19): Pyrrolysine
AGYLLGKLLXXLAAAALXXLL

SEQ ID NO: 3 - protein, 21 aa, Artificial Sequence
  PF6
  MISC_FEATURE (1)..(1): N-terminal modification Stearyl (C18)
  MISC_FEATURE (7)..(7): A lysine tree of three Lys residues is coupled to which 4 trifluoroquinoline molecules are attached
AGYLLGKINLKALAALAKKIL

SEQ ID NO: 4 - protein, 21 aa, Artificial Sequence
  PF14-OA
  MISC_FEATURE (1)..(1): N-terminal modification Oleyl (C181)
  MISC_FEATURE (10)..(11): Pyrrolysine
  MISC_FEATURE (18)..(19): Pyrrolysine
AGYLLGKLLXXLAAAALXXLL

SEQ ID NO: 5 - protein, 21 aa, Artificial Sequence
  PF14-IAH
  MISC_FEATURE (1)..(1): N-terminal modification Stearyl (C18)
  MISC_FEATURE (11)..(11): Pyrrolysine
  MISC_FEATURE (14)..(15): Pyrrolysine
  MISC_FEATURE (18)..(18): Pyrrolysine
AGYLLGKLLAXLAXXALXALL

SEQ ID NO: 6 - protein, 21 aa, Artificial Sequence
  PF14-4H
  MISC_FEATURE (1)..(1): N-terminal modification Stearyl (C18)
  MISC_FEATURE (10)..(11): Pyrrolysine
  MISC_FEATURE (18)..(19): Pyrrolysine
HHYHHGKLLXXLAAAALXXLL

SEQ ID NO: 7 - protein, 24 aa, Artificial Sequence
  PF14-6H
  MISC_FEATURE (1)..(1): Arachidyl (C20)
  MISC_FEATURE (13)..(14): Pyrrolysine
  MISC_FEATURE (21)..(22): Pyrrolysine
HHYHHYHHGKLLXXLAAAALXXLL

SEQ ID NO: 8 - protein, 21 aa, Artificial Sequence
  NF51
  MISC_FEATURE (1)..(1): N-terminal modification Stearyl (C18)
  MISC_FEATURE (7)..(7): ′X′ is Pyrrolysine. It is coupled to the side chain of ornithine (delta-amino group) instead of alpha-amino group
AGYLLGXINLKALAALAKKIL

SEQ ID NO: 9 - protein, 21 aa, Artificial Sequence
  NF55
  MISC_FEATURE (1)..(1): N-terminal modification Stearyl (C18)
  MISC_FEATURE (7)..(7): ′X′ is Pyrrolysine. It is coupled to the side chain of ornithine (delta-amino group) instead of alpha-amino group
AGYLLGXINLKALAALAKAIL

SEQ ID NO: 10 - protein, 23 aa, Artificial Sequence
  NF70
  MISC_FEATURE (1)..(1): N-terminal modification Arachidyl (C20)
  MISC_FEATURE (9)..(9): ′X′ is Pyrrolysine. It is coupled to the side chain of ornithine (delta-amino group) instead of alpha-amino group
HHHHYHHGXILLKALKALAKAIL

SEQ ID NO: 11 - protein, 21 aa, Artificial Sequence
  NF71
  MISC_FEATURE (1)..(1): N-terminal modification Stearyl (C18)
  MISC_FEATURE (7)..(7): ′X′ is Pyrrolysine. It is coupled to the side chain of ornithin (delta-amino group) instead of alpha-amino group
HHYHHGXILLKALKALAKAIL

SEQ ID NO: 12 - protein, 25 aa, Artificial Sequence
  NF72
  MISC_FEATURE (1)..(1): N-terminal modification Arachidyl (C20)
  MISC_FEATURE (11)..(11): ′X′ is Pyrrolysine. It is coupled to the side chain of ornithine (delta-amino group) instead of alpha-amino group
HHHHHHYHHGXILLKALKALAKAIL

SEQ ID NO: 13 - protein, 16 aa, Artificial Sequence
  WRAP1
LLWRLWRLLWRLWRLL

SEQ ID NO: 14 - protein, 15 aa, Artificial Sequence
  WRAP5
LLRLLRWWWRLLRLL

SEQ ID NO: 15 - protein, 20 aa, Artificial Sequence
  CADY
  MISC_FEATURE (1)..(1): N-terminal modification Acetyl (C2)
  MISC_FEATURE (20)..(20): C-terminus of peptide is cysteamide
GLWRALWRLLRSLWRLLWRA

SEQ ID NO: 16 - protein, 19 aa, Artificial Sequence
  hPeP3
  MISC_FEATURE (2)..(2): Alanine is (R)-2-(7-octenyl)alanine
  MISC_FEATURE (8)..(8): Alanine is (R)-2-(7-octenyl)alanine
  MISC_FEATURE (15)..(15): Alanine is (R)-2-(7-octenyl)alanine
LAKLAKAAAKLLKAAAKAL

SEQ ID NO: 17 - RNA, 18 nt, Artificial Sequence
  SCOs
ccucuuaccucaguuaca

SEQ ID NO: 18 - RNA, 18 nt, Artificial Sequence
  SCO 654
gcuauuaccuaaacccag